(19) United States
(12) Patent Application Publication
(10) Pub. No.: US 2005/0266412 A1
(43) Pub. Date: Dec. 1, 2005

(54) Method of amplifying infectious protein

(75) Inventor: Stanley B. Prusiner, San Francisco, CA (US)

Correspondence Address: BOZICEVIC, FIELD & FRANCIS LLP, 1900 UNIVERSITY AVENUE, SUITE 200, EAST PALO ALTO, CA, 94303, US

(73) Assignee: The Regents of the University of California

(21) Appl. No.: 10/854,742

(22) Filed: May 25, 2004

(51) Int. Cl. C12Q 1/68; G01N 33/53; C12P 21/06

(52) U.S. Cl. 435/6; 435/69.1; 435/7.1

(57) ABSTRACT

Infectious proteins such as prions present in a sample are amplified by adding a recombinant form (or portion thereof) of the infectious protein to the sample. The sample with the recombinant protein therein is maintained under cell free conditions which promote amplification for 20 hours or less and then assayed for the infectious protein.

### GOVERNMENT RIGHTS

The United States Government may have certain rights in this application pursuant to Grant Nos. AG02132, AG10880 and AG021601 awarded by the National Institutes of Health.

### FIELD OF THE INVENTION

This invention relates generally to the field of proteins and more specifically to the field of infectious proteins and methods of amplifying and detecting such proteins.

### BACKGROUND OF THE INVENTION

Some proteins can change from a normal configuration to an abnormal disease configuration. One such protein is PrP protein which can have a PrP^{C} normal configuration and a PrP_{Sc} disease configuration. The disease configuration is often referred to as a prion.

Prions are infectious pathogens that cause invariably fatal prion diseases (spongiform encephalopathies) of the central nervous system in humans and animals. Prions differ significantly from bacteria, viruses and viroids. The dominating hypothesis is that no nucleic acid is necessary to allow for the infectivity of a prion protein to proceed.

A major step in the study of prions and the diseases they cause was the discovery and purification of a protein designated prion protein [Bolton, McKinley et al. (1982) Science 218:1309-1311; Prusiner, Bolton et al. (1982) Biochemistry 21:6942-6950; McKinley, Bolton et al. (1983) Cell 35:57-62]. Complete prion protein-encoding genes have since been cloned, sequenced and expressed in transgenic animals. PrP^{C} is encoded by a single-copy host gene [Basler, Oesch et al. (1986) Cell 46:417-428] and when PrP^{C} is expressed it is generally found on the outer surface of neurons. Many lines of evidence indicate that prion diseases results from the transformation of the normal form of prion protein (PrP^{C}) into the abnormal form (PrP^{Sc}). There is no detectable difference in the amino acid sequence of the two forms. However, PrP^{Sc} when compared with PrP^{C} has a conformation with higher β-sheet and lower α-helix content [Pan, Baldwin et al. (1993) Proc Natl Acad Sci USA 90:10962-10966; Safar, Roller et al. (1993) J Biol Chem 268:20276-20284]. The presence of the abnormal PrP^{Sc} form in the brains of infected humans or animals is the only disease-specific diagnostic marker of prion diseases.

PrP^{Sc} plays a key role in both transmission and pathogenesis of prion diseases (spongiform encephalopathies) and it is a critical factor in neuronal degeneration [Prusiner (1997) The Molecular and Genetic Basis of Neurological Disease, 2nd Edition: 103-143]. The most common prion diseases in animals are scrapie of sheep and goats and bovine spongiform encephalopathy (BSE) of cattle [Wilesmith and Wells (1991) Curr Top Microbiol Immunol 172:21-38]. Four prion diseases of humans have been identified: (1) kuru, (2) Creutzfeldt-Jakob Disease (CJD), (3) Gerstmann-Streussler-Sheinker Disease (GSS), and (4) fatal familial insomnia (FFI) [Gajdusek (1977) Science 197:943-960; Medori, Tritschler et al. (1992) N Engl J Med 326:444-449]. Initially, the presentation of the inherited human prion diseases posed a conundrum which has since been explained by the cellular genetic origin of PrP.

Prions exist in multiple isolates (strains) with distinct biological characteristics when these different strains infect in genetically identical hosts [Prusiner (1997) The Molecular and Genetic Basis of Neurological Disease, 2nd Edition: 165-186]. The strains differ by incubation time, by topology of accumulation of PrP^{Sc} protein, and in some cases also by distribution and characteristics of brain pathology [DeArmond and Prusiner (1997) Greenfield's Neuropathology, 6th Edition:235-280]. Because PrP^{Sc} is the major, and very probably the only component of prions, the existence of prion strains has posed a conundrum as to how biological information can be enciphered in a molecule other than one comprised of nucleic acids. The partial proteolytic treatment of brain homogenates containing some prion isolates has been found to generate peptides with slightly different electrophoretic mobilities [Bessen and Marsh (1992) J Virol 66:2096-2101; Bessen and Marsh (1992) J Gen Virol 73:329-334; Telling, Parchi et al. (1996) Science 274:2079-2082]. These findings suggested different proteolytic cleavage sites due to the different conformation of PrP^{Sc} molecules in different strains of prions. Alternatively, the observed differences could be explained by formation of different complexes with other molecules, forming distinct cleavage sites in PrP^{Sc} in different strains [Marsh and Bessen (1994) Phil Trans R Soc Lond B 343:413-414]. Some researchers have proposed that different prion isolates may differ in the glycosylation patterns of prion protein [Collinge, Sidle et al. (1996) Nature 383:685-690; Hill, Zeidler et al. (1997) Lancet 349:99-100]. However, the reliability of both glycosylation and peptide mapping patterns in diagnostics of multiple prion strains is currently still debated [Collings, Hill et al. (1997) Nature 386:564; Somerville, Chong et al. (1997) Nature 386:564].

A system for detecting PrP^{Sc} by enhancing immunoreactivity after denaturation is provided in Serban, et al., Neurology, Vol. 40, No. 1, Ja 1990. Sufficiently sensitive and specific direct assay for infectious PrP^{Sc} in biological samples could potentially abolish the need for animal inoculations completely. Unfortunately, such does not appear to be possible with current PrP^{Sc} assays—it is estimated that the current sensitivity limit of proteinase-K and Western blot-based PrP^{Sc} detection is in a range of 1 μg/ml which corresponds to 10⁴-10⁵ prion infectious units. Additionally, the specificity of the traditional proteinase-K-based assays for PrP^{Sc} is in question in light of recent findings of only relative or no proteinase-K resistance of undoubtedly infectious prion preparations [Hsiao, Groth et al. (1994) Proc Natl Acad Sci USA 91:9126-9130] Telling, et al. (1996) Genes & Dev.

Human transthyretin (TTR) is a normal plasma protein composed of four identical, predominantly β-sheet structured units, and serves as a transporter of hormone thyroxine. Abnormal self assembly of TTR into amyloid fibrils causes two forms of human diseases, namely senile systemic amyloidosis (SSA) and familial amyloid polyneuropathy (FAP) [Kelly (1996) Curr Opin Strut Biol 6(1):11-7]. The cause of amyloid formation in FAP are point mutations in the TTR gene; the cause of SSA is unknown. The clinical diagnosis is established histologically by detecting deposits of amyloid in situ in biopsy material.

To date, little is known about the mechanism of TTR conversion into amyloid in vivo. However, several laboratories have demonstrated that amyloid conversion may be simulated in vitro by partial denaturation of normal human TTR [McCutchen, Colon et al. (1993) Biochemistry 32(45):12119-27; McCutchen and Kelly (1993) Biochem Biophys Res Commun 197(2) 415-21]. The mechanism of conformational transition involves monomeric conformational intermediate which polymerizes into linear β-sheet structured amyloid fibrils [Lai, Colon et al. (1996) Biochemistry 35(20):6470-82]. The process can be mitigated by binding with stabilizing molecules such as thyroxine or triiodophenol [Miroy, Lai et al. (1996) Proc Natl Acad Sci USA 93(26):15051-6].

In view of the above points, there is clearly a need for a specific, high flow-through, and cost-effective assay for testing sample materials for the presence of a pathogenic protein including transthyretin and prion protein.

In addition to PrP and TTR there are other proteins associated with other diseases.

The following is a non-limiting list of diseases with associated insoluble proteins which assume two or more different conformations.

| Disease | Insoluble Proteins |
|---|---|
| Alzheimer's Disease | APP, Aβ peptide, |
| | α1-antichymotrypsin, |
| | tan, non-Aβ component |
| Prion diseases, | PrP^{Sc} |
| Creutzfeld Jakob | |
| disease, scrapie and | |
| bovine spongeform | |
| encephalopathy | |
| ALS | SOD and neurofilament |
| Pick's disease | Pick body |
| Parkinson's disease | Lewy body |
| Diabetes Type 1 | Amylin |
| Multiple myeloma-- | IgGL-chain |
| plasma cell dyscrasias | |
| Familial amyloidotic | Transthyretin |
| polyneuropathy | |
| Medullary carcinoma | Procalcitonin |
| of thyroid | |
| Chronic renal failure | β₂--microglobulin |
| Congestive heart failure | Atrial natriuretic |
| | factor |
| Senile cardiac and | Transthyretin |
| systemic amyloidosis | |
| Chronic inflammation | Serum amyloid A |
| Atherosclerosis | ApoA1 |
| Familial amyloidosis | Gelsolin |

It should be noted that the insoluble proteins listed above each include a number of variants or mutations which result in different strains which are all encompassed by the present invention. Known pathogenic mutations and polymorphisms in the PrP gene related to prion diseases are given below and the sequences of human, sheep and bovine are given in U.S. Pat. No. 5,565,186, issued Oct. 15, 1996.

MUTATION TABLE

| Pathogenic human | Human | Sheep | Bovine |
|---|---|---|---|
| mutations | Polymorphisms | Polymorphisms | Polymorphisms |
| 2 octarepeat insert | Codon 129 | Codon 171 | 5 or 6 octarepeats |
| | Met/Val | Arg/Glu | |
| 4 octarepeat insert | Codon 219 | Codon 136 | |
| | Glu/Lys | Ala/Val | |
| 5 octarepeat insert | | | |
| 6 octarepeat insert | | | |
| 7 octarepeat insert | | | |
| 8 octarepeat insert | | | |
| 9 octarepeat insert | | | |
| Codon 102 Pro-Leu | | | |
| Codon 105 Pro-Leu | | | |
| Codon 117 Ala-Val | | | |
| Codon 145 Stop | | | |
| Codon 178 Asp-Asn | | | |
| Codon 180 Val-Ile | | | |
| Codon 198 Phe-Ser | | | |
| Codon 200 Glu-Lys | | | |
| Codon 210 Val-Ile | | | |
| Codon 217 Asn-Arg | | | |
| Codon 232 Met-Ala | | | |

When these proteins are present in very small amounts the individual does not exhibit symptoms of disease. It would be desirable to know that small amounts of the disease form of the protein are present if only to prevent passing the infection on to another individual. However, current assays can not, in general, detect the protein below a certain level. Thus, there is a need for a method whereby small amounts of infectious protein present in a sample can be amplified. The present invention provides such.

### SUMMARY OF THE INVENTION

A method is disclosed whereby the amount of infectious protein present in a sample is amplified. The method comprises adding a recombinantly produced protein or portion thereof (corresponding to the protein to be amplified) to a sample which may contain the disease form of the protein to be amplified. Conditions promoting amplification are maintained (in vitro) over a limited period of time after which the sample is tested for the presence of the disease form of the protein. Infectious proteins being tested for are those generally associated with neurodegenerative diseases including but not limited to prion diseases, e.g. Parkinson's, and Alzheimer's. Thus, by detecting proteins associated with the disease in a sample it is possible to enhance the accuracy of diagnosing the disease in a patient from which the sample was extracted.

An aspect of the invention is that recombinantly produced proteins in a non-disease conformation can be used to increase the amount of a disease conformation of a protein in a sample.

Another aspect of the invention is that recombinantly produced portion(s) of a protein of interest can be added to a sample containing a disease conformation of that protein to increase the amount of the disease conformation of the protein in the sample.

Yet another aspect of the invention is that the recombinantly produced protein or portion thereof may be any animal protein, e.g. mammalian protein, e.g. human or cow protein that assumes both a normal and a disease conformation.

Still another aspect of the invention is that the protein amplification methodology can be used to prepare a sample for assaying in any type of assay by amplifying the protein of interest in a sample being tested.

Still yet another aspect of the invention is that it can be used on any type of sample including, brain tissue, nerve cells, muscle tissue, blood, cells and tissue used in transplantation, etc. in order to enhance the sensitivity of any assay used on such to detect infectious proteins.

These and other aspects, advantages, and objects of the invention will become apparent to those persons skilled in the art upon reading this disclosure in combination with the figures attached hereto.

### BRIEF DESCRIPTION OF THE DRAWINGS

The invention is best understood from the following detailed description when read in conjunction with the accompanying drawings. It is emphasized that, according to common practice, the various features of the drawings are not to-scale. On the contrary, the dimensions of the various features are arbitrarily expanded or reduced for clarity. Included in the drawings are the following figures:

FIG. 1 is a graph showing prion disease incubation times for animals (a) inoculated with seeded PrP proteins (black squares), (b) unseeded recombinant protein (black diamond) and (c) uninnoculated animals.

FIG. 2A is an image of an immunoblot of labeled PrP^{Sc} in brains of Tg(MoPrP, Δ23-88) 9949/Prnp^{0/0} mice and FIG. 2B is an image of an immunoblot of labeled PrP^{Sc} in brains of Tg(MoPrP) 4053, wild-type CD1 and FVB mice.

FIG. 3 shows six photos labeled, A, B, C, D, E and F of animal brain slices showing neuropathological features of the brain tissue in both seeded and unseeded animals.

FIG. 4A is a graph showing survival times for FVB mice inoculated with RML (open triangle) and inoculated with SMP1 (solid diamond). FIG. 4B is a graph showing survival times for Tg (MoPrP) 4053 mice inoculated with RML (open triangle) and inoculated with SMP1 (solid diamond).

FIG. 5 shows six photos labeled A, B, C, D, E and F of animal brain slices showing differences in neuropathological changes between Tg 9949 mice (A, B and C) inoculated with seeded recombinant PrP and FVB mice (D, E and F) inoculated with second passage of seeded preparations derived from homogenized brains of clinically ill Tg 9949 mice.

FIG. 6 is an image of an immunoblot with three lanes where lane M shows molecule weight markers, lane I shows wild-type recombinant MoPrP (89-230), and lane 2 shows wild-type recombinant MoPrP (23-231).

FIG. 7 is a graph showing the detected amount of fluorescence over time for 40 hours where the open squares are for a seeded samples of recombinant MoPrP (89-230) and the blackened circles are for unseeded recombinant MoPrP (89-230).

FIG. 8 is an electron micrograph of amyloid fibrils of a type used for seeding a sample in connection with the present invention.

FIG. 9 is a graph showing vasculation scores for TgH9949 mice for different types of brain tissue for both unseeded (light bars) and seeded (black bars) with recMoPrP.

FIG. 10 is a graph showing vasculation scores for TgH9949 mice inoculated with RML prions.

FIG. 11 is a bar graph of vasculation scores on different areas of the brain for Tg4053 mice inoculated with seeded recombinant MoPrP prions.

FIG. 12 is a bar graph of vasculation scores of different areas of the brain for Tg 4053 mice inoculated with RML prions.

### DETAILED DESCRIPTION OF THE INVENTION

Before the present protein amplification method and reagent used therewith are described, it is to be understood that this invention is not limited to particular embodiment or proteins described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates. otherwise, between the upper and lower limits of that range is also specifically disclosed. Each smaller range between any stated value or intervening value in a stated range and any other stated or intervening value in that stated range is encompassed within the invention. The upper and lower limits of these smaller ranges may independently be included or excluded in the range, and each range where either, neither or both limits are included in the smaller ranges is also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, the preferred methods and materials are now described. All publications mentioned herein are incorporated herein by reference to disclose and describe the methods and/or materials in connection with which the publications are cited.

It must be noted that as used herein and in the appended claims, the singular forms “a”, “an”, and “the” include plural referents unless the context clearly dictates otherwise. Thus, for example, reference to “a protein” includes a plurality of such proteins and reference to “the reagents” includes reference to one or more reagents and equivalents thereof known to those skilled in the art reading this disclosure, and so forth.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed.

### THE INVENTION IN GENERAL

The present invention shows, for the first time, that it is possible to make an infectious protein in vitro in a cell free system. The proteins made have been shown to be infectious by inoculating transgenic mice with proteins produced. Because methods shown here “seed” the reagent with amyloid fibrils those skilled in the art reading this disclosure will understand that the “seeding” can be replaced with infectious proteins present in a sample to be tested and that the sample may be treated. When the sample contains the “seed” or infectious protein that protein will be amplified or produced many times over. Such amplification increases the sensitivity of assays to detect the presence of infectious proteins.

In it's simplest form the invention involves combining a reagent with a sample and maintaining the combination under conditions which allow for amplification of any infectious proteins present in the sample. The reagent is comprised of a recombinant protein, or a portion of a recombinant protein such as a significant C-terminal portion of a protein which corresponds to the amino acid sequence of the native protein being amplified. The method is carried out in vitro and in particular in a cell free system. The amplification is carried out and the assay is the run thereafter in a period of time less than 40 hours, preferably less than 20 hours, and may be in increments of 8, 7, 6, 5, 4, 3, 2, or 1 hour or less in order to obtain the desired amplification of any infectious protein in the sample. An important aspect of the invention is that the resulting amplified protein is shown to be infectious when used to inoculate an animal. Specifically, if the amplified protein is a human PrP protein and specifically a human prion when that human prion is used to inoculate a transgenic mouse which has a human PrP gene therein the mouse will become sick showing distinct evidence of a prion disease.

Although the invention is described here in connection with the PrP proteins and prion diseases those skilled in the art reading this disclosure will understand that the invention can be used in connection with proteins associated with other diseases wherein the protein is an infectious protein, i.e. a protein which assumes a first normal configuration and a second disease related conformation wherein both conformations have the same amino acid sequence. Examples of such diseases and their associated insoluble proteins are described in the background of this disclosure.

Specific methods of carrying out amplification using very specific reagents and conditions are described here. Those skilled in the art will understand that a degree of variation is permitted and that such is still within the scope of the present invention. Within the examples preparations are “seeded” with amyloid fibrils. However, to carry out the method of the invention on a sample the amyloid fibrils will be the infectious proteins present within the sample. If no infectious proteins are present then no amplification will occur in the time allowed for (see FIG. 7) and the assay used on the resulting sample will show negative. However, those skilled in the art reading this disclosure will recognize that it will be desirable to prepare the sample prior to carrying out the amplification method of the present invention.

The sample preparation methodology may involve concentration of any prions or other insoluble proteins which might be present in the sample. Sample concentration can be carried out by adding to the sample a binding agent such as phosphotungstic acid or a salt thereof which binds to the insoluble form of such proteins such as PrP^{Sc}. The binding agent is one such that when the binding agent to the protein alone. Thus, the combination can be subjected to a centrifuge in order to concentrate the protein bound to the binding agent and the concentrate can be tested. Such methods of sample preparation are described within U.S. Pat. No. 5,977,324 issued Nov. 2, 1999. It will also be understood by those skilled in the art reading this disclosure that the sample may be subjected to other processing such as by contacting the sample with enzymes which will cleave away portions of the insoluble protein leaving only a distinct insoluble core, e.g. PrP27-30 as described within U.S. Pat. No. 5,977,324.

In addition to sample preparation methods as described above those skilled in the art reading this disclosure will understand that certain samples require very specific preparation. For example, it is difficult to detect insoluble proteins such as insoluble PrP proteins within blood. To obtain a positive reading in a blood sample which has infectious proteins therein it is generally necessary to allow the blood to clot and then separate the serum away from the clotted blood. The serum is then contacted with a complexing agent of the type as described above in order to form a complex an then concentrate the complex to carry out the assay. Methods of this type are described within U.S. Pat. No. 6,166,187 issued Dec. 26, 2000.

The methodology of the present invention does not merely produce proteins. An important aspect of the invention is that the proteins produced are “infectious” in that they are capable of causing disease in an animal. Infectious proteins produced in accordance with the methodology disclosed herein have been tested in transgenic animals in order to confirm that they are infectious. Others can confirm such by using transgenic animals such as the transgenic mice disclosed and described within U.S. Pat. No. 5,792,901. Further, those animals can be used in controlled studies using standard prion preparations of the type described within U.S. Pat. No. 6,020,537 issued Feb. 1, 2000.

Once the method of the invention has been carried out in order to produce infectious proteins, i.e. amplify the amount of infectious proteins in the sample the sample can be further prepared as described above and can be assayed for the presence of such infectious proteins. It is possible to use essentially any assay known to test for infectious proteins such as the conformation dependent immunoassay (CDI) of the type described within U.S. Pat. No. 5,891,641 issued Apr. 6, 1999. It is also possible to confirm the presence of the infectious proteins using antibodies such as the antibodies disclosed and described within U.S. Pat. No. 5,846,533 issued Dec. 8, 1998. It may be desirable to use specific antibodies such as when assaying bovine brain for prions which infect cows. A specific antibody useful for assaying ungulates is disclosed within U.S. Pat. No. 6,537,548 issued Mar. 25, 2003.

To demonstrate the amplification methodology of the present invention an experiment was carried out to refold wt MoPrP(89-230) into amyloid fibrils and bioassay those fibrils in mice expressing the corresponding PrP. The PrP amyloid represents a limited subset of β-rich PrPs, all of which are infectious. It is important to note that PrP amyloid deposition is a nonobligatory constituent of prion diseases (S. B. Prusiner et al., Cell 63, 673-686 (1990)), in contrast to some other disorders in which amyloids seem to be constant features (C. M. Dobson, Nature 426, 884-890 (2003)).

Because PrP 27-30 polymerizes into amyloid fibrils (S. B. Prusiner et al., Cell 35, 349-358 (1983); M. P. McKinley et al., J. Virol. 65, 1340-1351 (1991)) and full-length PrP^{Sc} does not, N-terminally truncated MoPrP composed of residues 89-230 was expressed in E. coli. This protein, denoted recMoPrP(89-230) or recMoPrP(Δ23-88), was purified to homogeneity and folded into a β-sheet-rich state that assembled into amyloid fibrils which fibrils are shown in the electron micrograph of FIG. 8. Two protocols were used to produce the fibrils. One protocol used monomeric recMoPrP(Δ23-88) to produce the amyloid fibrils that are referred to as “unseeded” (I. V. Baskakov, G. Legname, M. A. Baldwin, S. B. Prusiner, F. E. Cohen, J. Biol. Chem. 277, 21140-21148 (2002)). A second protocol used some of the unseeded fibrils as a seed for the production of nascent fibrils, which are denoted as “seeded”.

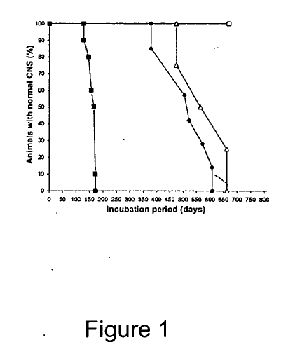

After producing both seeded and unseeded amyloid fibrils composed of recMoPrP(Δ23-88), Tg(MoPrP, Δ23-88)9949/Prnp^{0/0} mice, hereafter referred to as Tg9949 mice were inoculated. The Tg9949 mice express MoPrP(Δ23-88) at a level 16-fold greater than SHaPrP in Syrian hamsters (S. Supattapone et al., J. Virol. 75, 1408-1413 (2001)). The Tg9949 mice received intracerebrally either unseeded or seeded amyloid preparations and were followed for clinical signs of nervous system dysfunction. All of the mice developed neurologic disease between 380 and 660 days after inoculation (see FIG. 1 and the Table below).

TABLE
Transmission of synthetic and natural prion strains to Tg9949 mice

| Mouse strain | | | | |
|---|---|---|---|---|
| (expression | | Incubation time | | |
| level) | Inoculum | (days ± SEM) | Passage | n/n₀^{b} |
| Tg9949^{a} (16×) | Seeded/recMoPrP | 516.3 ± 71.5 | 1st | 7/7 |
| | Unseeded/recMoPrP | 590.8 ± 90.6 | 1st | 4/4 |
| | none | >670* | | 0/7 |
| | RML | 159.9 ± 12.4 | 1st | 11/11 |
| | (9949)RML | 142.7 ± 21.5 | 2nd | 10/10 |
| | Me7 | 219.9 ± 11.5 | 1st | 7/7 |
| | 301V | 433 ± 49.8 | 1st | 4/4 |
| | C506 | 252.8 ± 93.4 | 1st | 10/10 |
| | 139H | 479 ± 24.5 | 1st | 3/3 |
| | DY | 143 ± 15.3 | 1st | 8/8 |
| FVB (1×) | (9949) | 153.9 ± 11.1 | 1st | 9/9 |
| | Seeded/recMoPrP | | | |
| | RML | 116.5 ± 9.5 | 1st | 10/10 |
| Tg4053^{a} (8×) | (9949) | 89.7 ± 3.3 | 1st | 10/10 |
| | Seeded/recMoPrP | | | |
| | RML | 55.3 ± 7.5 | 1st | 10/10 |

^{a}All transgenes are expressed in Prnp^{0/0} mice. Expression levels of PrP^{C} relative to normal SHaPrP levels in hamster brain were determined by immunoblots of serially diluted brain homogenates.
^{b}Number of animal developing prion disease/total number.
*Tg9949 mice did not show any signs of neurologic dysfunction over 670 days of age, at which time they were sacrificed. An additional uninocluated Tg9949 mouse was sacrificed at 580 days of age and failed to show any vacuolation or PrP deposits on neuropathologic evaluation or any protoease-resistant PrP on Western blotting.

The mice inoculated with seeded amyloid exhibited shorter incubation times compared to those with unseeded amyloid. Seven uninoculated Tg9949 mice remained healthy for 670 days and were sacrificed after the last amyloid-inoculated Tg9949 mice developed illness. In earlier studies, uninoculated Tg9949 mice lived for more than 500 days without any signs of neurologic dysfunction (S. Supattapone et al., J. Virol. 75, 1408-1413 (2001)).

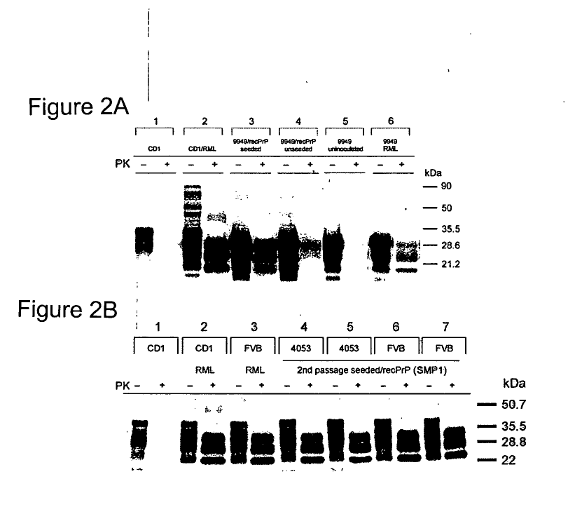

The shortest incubation time for a Tg9949 mouse inoculated with seeded amyloid was 382 days compared to 474 days for a Tg9949 mouse inoculated with unseeded amyloid. Western blot analysis of brain homogenates of these two mice revealed that the Tg9949 mouse inoculated with seeded amyloid had more protease-resistant PrP than the brain of the unseeded amyloid-inoculated mouse (see FIG. 2A).

Whether the different incubation times and diverse biochemical profiles reflect higher levels of PrP^{Sc} in the seeded amyloid compared to the unseeded or the creation of two different prion strains remains to be established. No protease-resistant PrP was found by Western blotting in the brain of an uninoculated Tg9949 mouse sacrificed at 580 days of age (FIG. 2A). No protease-resistant PrP could be detected in either the seeded nor unseeded amyloid preparations (I. V. Baskakov, G. Legname, M. A. Baldwin, S. B. Prusiner, F. E. Cohen, J. Biol. Chem. 277, 21140-21148 (2002)). The level of MoPrP^{Sc}(89-230) in the fibril, if present, was too low to be detected by Western blotting. Whether the amyloid fibrils protected the small amounts of PrP^{Sc} found within them or modified the retention of PrP^{Sc} in brain after inoculation remains to be determined. Greater than 90% of bacteriophage and India ink particles are washed out of the brains of mice inoculated intracerebrally (R. W. Schlesinger, J. Exp. Med. 89, 491-505 (1949); H. J. F. Cairns, Nature 166, 910 (1950)).

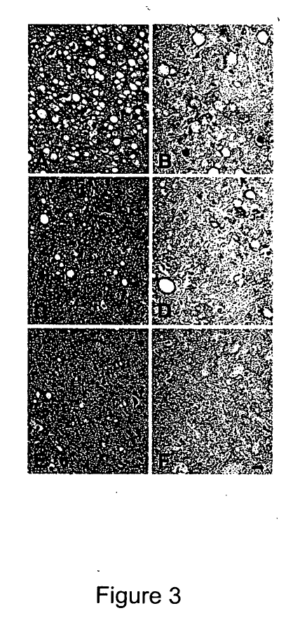

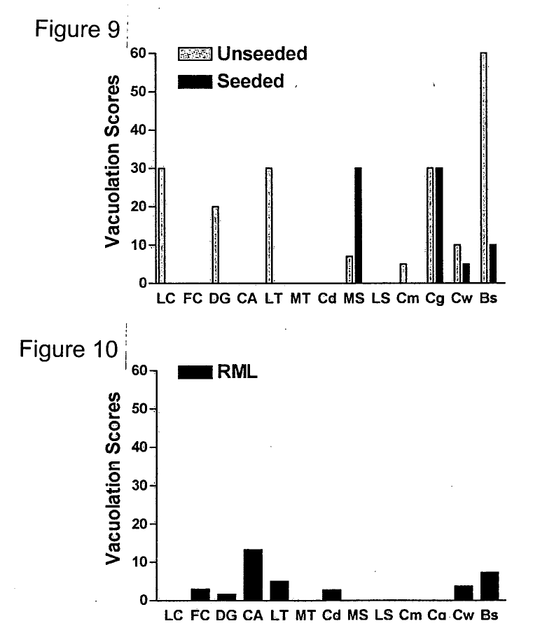

Neuropathological examination of Tg9949 mice inoculated with seeded synthetic prions revealed extensive vacuolation with associated gliosis in the cerebellum, hippocampus, brainstem and white matter (FIGS. 3B and E). The distribution, density, and morphology of the vacuoles associated with the unseeded and seeded amyloid preparations were different, raising the possibility that they represent two different prion strains (FIGS. 3A and B). Vacuolation, astrocytic gliosis, and PrP^{Sc} accumulation were more widely dispersed in gray matter regions in an animal inoculated with unseeded amyloid compared to animals inoculated with seeded amyloid (FIGS. 3D and 3E). The neuroanatomic distributions of vacuoles associated with unseeded and seeded amyloid were different from those found with RML prions (compare FIGS. 3A and 3B with 3C). It is also pointed out that the sizes of vacuoles resulting from each inoculum were different. From unseeded amyloid preparations, the majority of vacuoles measured 20 to 50 μm in diameter (see FIG. 9), whereas most vacuoles from RML prions were 10 to 30 μm in diameter (see FIG. 10). From the seeded amyloid inoculum, smaller (10 to 20 μm) and larger (20 to 50 μm) vacuoles were evenly represented (see FIG. 10). With both unseeded and seeded amyloid, PrP^{Sc} deposited in gray matter as relatively large solitary masses of 5 to 20 μm in diameter and formed a perimeter at the edge of the vacuoles. In contrast, these PrP^{Sc} deposits from RML infection consisted of finely granular PrP^{Sc} accumulations.

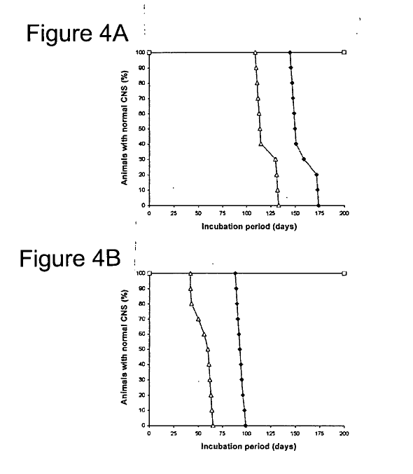

Prions in the brains of Tg9949 mice that had been inoculated with seeded amyloid were designated “synthetic mammalian prion strain 1,” or SMP 1. Serial transmission of SMP1 prions from Tg9949 mice to wt FVB and Tg(MoPrP-A)4053 mice gave mean incubation times of 154 and 90 days, respectively (see FIGS. 4A and 4B and the above Table). The Tg(MoPrP-A)4053 mice express MoPrP-A at a level 8-fold greater than SHaPrP in Syrian hamsters (15) and are denoted Tg4053 mice below. Wt FVB and Tg4053 mice inoculated with RML prions exhibited incubation times of 116 and 55 days, respectively. Biochemical analysis of brain homogenates from second passage of SMP1 prions in wt FVB and Tg4053 confirmed the presence of protease-resistant PrP^{Sc}, indicating the efficient transmission of infectivity between passages (see FIG. 2B).

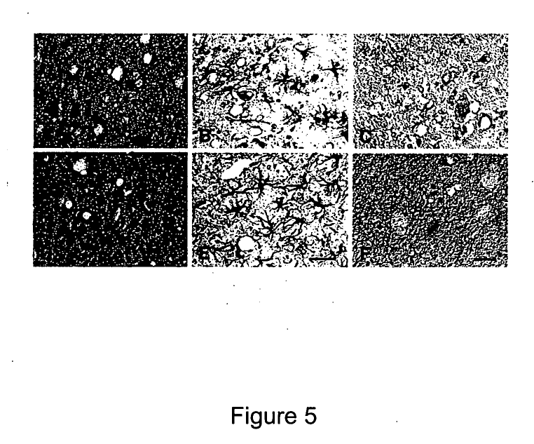

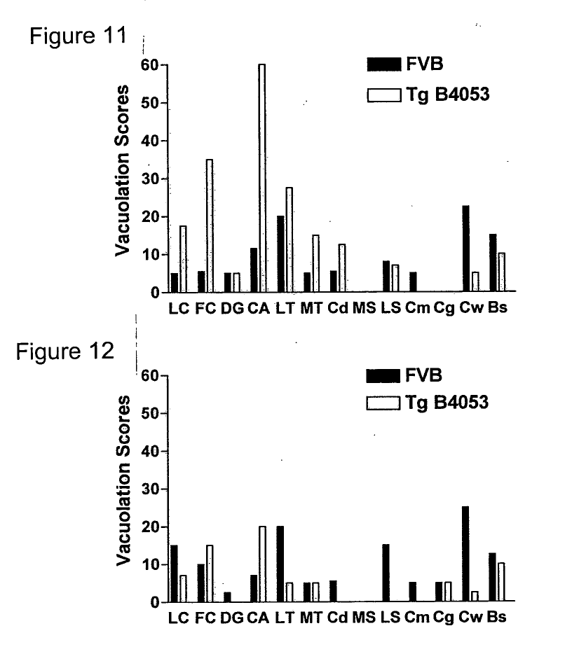

Well-defined PrP amyloid plaques as well as numerous, densely packed, finely granular PrP^{Sc} deposits were identifiable in second passage in both FVB (FIG. 5F) and Tg4053 mice. The vacuolation scores (the percentage of an area occupied by vacuoles) were greater in Tg4053 than FVB mice (See FIG. 11). Importantly, the density of vacuoles (number per area) was greater for SMP1 than for RML prions in Tg4053 mice (See FIG. 12). Moreover, RML prions failed to cause vacuolation in the caudate nucleus, septal nuclei, and cerebellar white matter in Tg4053 mice. This shows that the characteristics of the SMP1 strain remained stable during passage from Tg9949 to Tg4053 mice. The characteristics of the SMP1 strain were less stable on passage in FVB mice, showing that SMP1 and RML prions share several features. Combined with the widespread immunostaining for PrP^{Sc} deposition (see FIG. 5F), these results show that synthetic prions in the seeded amyloid adopted some features similar to RML prions on passage in FVB mice.

The present invention is directed at producing synthetic prions in vitro using the formation of PrP amyloid as a surrogate marker for the folding of MoPrP(89-230) into a biologically active conformation. The rapidity and ease of measuring thioflavin T binding that reflects amyloid formation (J. H. Come, P. E. Fraser, P. T. Lansbury, Jr., Proc. Natl. Acad. Sci. USA 90, 5959-5963 (1993); H. LeVine, Protein Sci. 2, 404410 (1993)) facilitate at the ability to determine conditions under which recMoPrP(89-230) assembles into amyloid fibrils (I. V. Baskakov, G. Legname, M. A. Baldwin, S. B. Prusiner, F. E. Cohen, J. Biol. Chem. 277, 21140-21148 (2002)). The results provided here show that such fibrils harbor detectable levels of prion infectivity making it possible to draw a series of conclusions about mammalian prions that were previously elusive.

First, the results provided here show that the prion protein is both necessary and sufficient for infectivity; prions are infectious proteins.

Second, neither the Asn-linked oligosaccharides nor the glycosylphosphatidylinositol anchor are required for prion infectivity since the recMoPrP(89-230) used in the experiments described here contains neither of these posttranslational modifications (A. Taraboulos et al., Proc. Natl. Acad. Sci. USA 87, 8262-8266 (1990); S. J. DeArmond et al., Neuron 19, 1337-1348 (1997); P. Gambetti, P. Parchi, N. Engl. J. Med. 340, 1675-1677 (1999); J. A. Mastrianni et al, N. Engl. J. Med. 340, 1630-1638 (1999)).

Third, the biological information carried by distinct strains of prions resides in PrP^{Sc}. Moreover, variations in PrP glycosylation are not required for prion diversity.

Fourth, the spontaneous formation of prions, which is responsible for sporadic forms of prion disease in livestock and humans, can occur in any mammal expressing PrP^{C}.

The results provided here show that prion diseases are disorders of protein conformation in which PrP^{C} and PrP^{Sc} represent distinct structural states. Previous difficulties in creating in vitro infectious prions from recPrPs enriched for β-structure may be due the tendency of mammalian PrPs to fold into biologically irrelevant β-rich isoforms. Although the strategy used in the experiments described here may appear rather straightforward in retrospect, the use of recombinant PrP in the method described here eluded researches for many years.

From Tg mouse studies, it is well established that templates improve the likelihood of forming an infectious β-rich isoform (S. Supattapone et al., J. Virol. 75, 1408-1413 (2001); S. B. Prusiner et al., Cell 63, 673-686 (1990)). The results provided here show that “seeded” amyloid fibrils exhibit shorter incubation times than their “unseeded” progenitor (see FIG. 1). These results show that “cell-free conversion assays” (D. A. Kocisko et al., Nature 370, 471-474 (1994)) and “cell-free amplification systems” (G. P. Saborio, B. Permanne, C. Soto, Nature 411, 810-813 (2001); N. R. Deleault, R. W. Lucassen, S. Supattapone, Nature 425, 717-720 (2003)) can be improved to increase the yield of the infectious β-rich isoform. In the past, it has been difficult to judge the utility of these methods owing to the requirement for small amounts of biologically derived PrP^{Sc}.

A bona fide cell-free amplification system for infectious proteins such as prions would be valuable in assaying the safety of a range of foods including beef, lamb, pork and chicken as well as a biological material obtained from a patient to treat another patient such as blood, blood products, cells, tissues, organs, etc. Thus, these results have important implications for human health. The formation of prions from recPrP demonstrates that PrP^{C} is sufficient for the spontaneous formation of prions, and thus, no exogenous agent is required for prions to form in any mammal. The results shown here provide an explanation for sporadic Creutzfeldt-Jakob disease for which the spontaneous formation of prions has been hypothesized (S. B. Prusiner, Annu. Rev. Microbiol. 43, 345-374 (1989)). Understanding sporadic prion disease is particularly relevant to controlling the exposure of humans to bovine prions (A. G. Biacabe, J. L. Laplanche, S. Ryder, T. Baron, EMBO Rep 5, 110-115 (2004); C. Casalone et al., Proc Natl Acad Sci USA 101, 3065-3070 (2004); Y. Yamakawa et al., Jpn J Infect Dis 56, 221-222 (2003)). That bovine prions are pathogenic for humans is well documented in the cases of more than 150 teenagers and young adults who have already died from prion-tainted beef derived from cattle with bovine spongiform encephalopathy (BSE) (R. G. Will et al., Lancet 347, 921-925 (1996); M. R. Scott et al., Proc. Natl. Acad. Sci. USA 96, 15137-15142 (1999); R. G. Will, M. P. Alpers, D. Dormont, L. B. Schonberger, in Prion Biology and Diseases S. B. Prusiner, Ed. (Cold Spring Harbor Laboratory Press, Cold Spring Harbor, 2004) pp. 629-671). Moreover, the sporadic forms of Alzheimer's and Parkinson's diseases as well as amyotrophic lateral sclerosis and the frontal temporal dementias are the most frequent forms of these age-dependent disorders as is the case for the prion diseases (L. E. Hebert, P. A. Scherr, J. L. Bienias, D. A. Bennett, D. A. Evans, Arch Neurol 60, 1119-1122 (2003)). Important insights in the etiologic events that feature in these more common neurodegenerative disorders, all of which are caused by the aberrant processing of proteins in the nervous system, are likely to emerge as more is learned about the molecular pathogenesis of sporadic prion diseases (S. B. Prusiner, N. Engl. J. Med. 344, 1516-1526 (2001)).

### EXAMPLES

The following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Centigrade, and pressure is at or near atmospheric.

### Example 1

Amyloid fibrils were formed upon incubation of recMoPrP(89-230) (0.6 mg/ml) at 37° C. in 3 M urea, 0.2 M NaCl, 50 mM Na-acetate buffer, pH 5.0 as previously described (I. V. Baskakov, G. Legname, M. A. Baldwin, S. B. Prusiner, F. E. Cohen, J. Biol. Chem. 277, 21140-21148 (2002)). The kinetics of fibril formation were monitored using a thioflavin T binding assay (H. LeVine, Protein Sci. 2, 404-410 (1993)). Inocula were prepared by dialysis of the fibrils in PBS buffer, pH 7.2 for two days. Approximate concentration of recMoPrP(89-230) in the inocula was 0.5 mg/ml. FIG. 1 shows survival curves for three groups of Tg(MoPrP,Δ23-88)9949/Prnp^{0/0} mice inoculated with RML prions (▪), seeded-amyloid recMoPrP (♦) and unseeded-amyloid recMoPrP(89-230) (Δ). Uninoculated mice (□) did not show any clinical symptoms up to 670 days of age, at which time they were sacrificed.

FIG. 2A shows images of immunoblot of PrP^{Sc} in brains of Tg(MoPrP, Δ23-88)9949/Prnp^{0/0} mice. In FIG. 2A the six paired sample lanes are numbered: (1) uninoculated, normal CD1 mouse, (2) RML-inoculated CD1 mouse, (3) Tg(MoPrP,Δ23-88)9949/Prnp^{0/0} mice inoculated with seeded-amyloid recPrP, (4) Tg(MoPrP,Δ23-88)9949/Prnp^{0/0} mice inoculated with unseeded-amyloid recPrP and (5) uninoculated Tg(MoPrP,Δ23-88)9949/Prnp^{0/0} mice and sacrificed at 580 days of age. FIG. 2B shows images of immunoblot of PrP^{Sc} in brains of Tg(MoPrP)4053, wild-type CD1 and FVB mice. In FIG. 2B the seven paired samples lanes are numbered: (1) uninoculated, normal CD1 mouse, (2) RML-inoculated CD1 mouse, (3) RML-inoculated FVB mouse, (4 and 5) Tg(MoPrP)B4053 mice inoculated with brain homogenate from Tg(MoPrP,Δ23-88)9949/Prnp^{0/0} mice inoculated with seeded-amyloid recMoPrP (2nd passage), (6 and 7) FVB mice inoculated with brain homogenate from Tg(MoPrP,Δ23-88)9949/Prnp^{0/0} mice inoculated with seeded-amyloid recMoPrP (2^{nd} passage). Minus (−) symbol denotes undigested control sample, and plus (+) symbol designates samples subjected to limited proteolysis using proteinase K (PK). Apparent molecular weights based on migration of protein standards are given in kilodaltons (kDa).

FIG. 3 provides six photo images showing distinguishing neuropathological features of unseeded recPrP prions (A, B), seeded recPrP prions (C, D), and RML prions (E, F) in the pons of TgH9949 mice. (A, C, E) H&E stain. (B, D, F) Immunohistochemistry of PrP^{Sc} by the hydrated autoclaving method using the PrP-specific HuM-R2 monoclonal antibody (D. Peretz et al., Nature 412, 739-743 (2001)). Bar in E is 50 μm and also applies to A and C. Bar in F is 25 μm and also applies to B and D.

FIG. 4A shows a graph with the survival curves of FVB mice inoculated with RML (Δ) and SMP1 (♦) prions. Uninoculated mice (□) did not show any clinical symptoms up to 200 days of age, at which time they were sacrificed. FIG. 4B shows a graph with the survival curves of Tg(MoPrP)4053 mice inoculated with RML (Δ) and SMP1 (♦) prions. Uninoculated mice (□) did not show any clinical symptoms up to 200 days of age, at which time they were sacrificed.

FIG. 5 provides six photographic images providing a comparison of neuropathological changes in the pons associated with primary inoculation of seeded recPrP preparations into Tg9949 mice (A, B, C) and with second passage of seeded preparations derived from clinically ill Tg9949 mice inoculated into FVB mice (D, E, F). Both passages show the neurohistological characteristics of a prion disease: Vacuoles (spongiform degeneration), H&E stain (A and D); reactive astrocytic gliosis, GFAP immunohistochemistry (B and E); and accumulation of PrP^{Sc}, hydrated autoclaving immunohistochemistry with the PrP-specific R2 monoclonal antibody (C and F). Bar in E is 50 μm and also applies to A, B, and D. Bar in F is 25 μm and also applies to C.

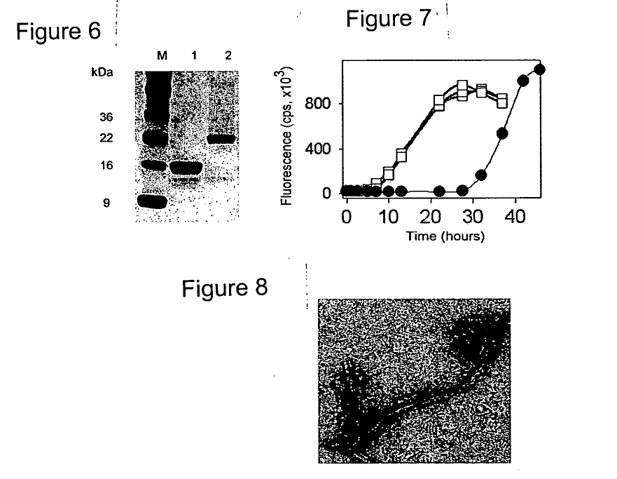

FIG. 6 is an image of an immunoblot provided to show expression and refolding of recombinant MoPrP(89-230). Expressed and purified recombinant PrPs (I. Mehlhorn et al., Biochemistry 35, 5528-5537 (1996)) were separated in 16% Tris-glycine SDS-PAGE gel (Invitrogen) and silver stained. Lane M of FIG. 6 was used for protein molecular weight markers. Lane 1 of FIG. 6 was for wild-type recombinant MoPrP(89-230) and Lane 2 was for wild-type recombinant MoPrP(23-23 1) and is shown for comparison. Molecular weight markers are expressed in kilodaltons (kDa). Mass spectrometry measurements for full-length recMoPrP(23-230) and the N-terminally truncated recMoPrP(89-230) were made and compared to the theoretical mass.

To obtain the data for the graph of FIG. 7 recMoPrP(89-230) (0.5 mg/ml) in 0.6 ml was incubated at 37° C. in 3 M urea, 0.2 M NaCl, 50 mM Na-acetate buffer, pH 5.0 using a conical shaker oscillating at 600 rpm (I. V. Baskakov, G. Legname, M. A. Baldwin, S. B. Prusiner, F. E. Cohen, J. Biol. Chem. 277, 21140-21148 (2002)). Seeded PrP amyloid fibrils were prepared using the same conditions as those used for the unseeded fibrils except 1% (w/w) of freshly prepared, preformed fibrils composed of recMoPrP(89-230) was added to the reaction mixture. Kinetics of amyloid formation for unseeded recMoPrP(89-231) (filled circles) and seeded (open squares) were monitored using the thioflavin T binding assay (H. LeVine, Protein Sci. 2, 404-410 (1993)). Inocula (0.5 mg/ml) for bioassays were prepared by dialysis of 2 ml of PrP fibrils using 2 L of stirred PBS buffer, pH 7.2 that was changed 3 times over 2 days.

FIG. 8 is an electron micrograph of amyloid fibrils formed from recMoPrP(89-230) negatively stained with ammonium molybdate.

FIGS. 9 and 10 are each bar graphs of the vacuolation score histograms from TgH9949 mouse brains show that vacuolation phenotype is different for the three inoculates. FIG. 9 shows both unseeded and seeded recMoPrP prions and FIG. 10 shows results for RML prions. The vacuolation histogram is a semiquantitative estimate of the area of a brain region occupied by vacuoles. Bs, brainstem (pons); CA, cornu ammonis of the hippocampus; Cd, caudate nucleus; Cg, cerebellar granule cell layer; Cm, cerebellar molecular layer; Cw, cerebellar white matter; DG, dentate gyrus of the hippocampus; FC, frontal cortex; LC, limbic cortex (cingulate gyrus); LS, lateral septal nuclei; LT, lateral thalamic nuclei; MS, medial septal nuclei; MT, medial thalamic nuclei.

FIGS. 11 and 12 are each bar graphs of data of vacuolation score histograms from FVB and Tg4053 mice. FIG. 11 is of mouse brain inoculated with seeded recMoPrP prions and FIG. 12 is from mice inoculated with RML prions. The areas from which the data were obtained are as in FIGS. 9 and 10.

The preceding merely illustrates the principles of the invention. It will be appreciated that those skilled in the art will be able to devise various arrangements which, although not explicitly described or shown herein, embody the principles of the invention and are included within its spirit and scope. Furthermore, all examples and conditional language recited herein are principally intended to aid the reader in understanding the principles of the invention and the concepts contributed by the inventors to furthering the art, and are to be construed as being without limitation to such specifically recited examples and conditions. Moreover, all statements herein reciting principles, aspects, and embodiments of the invention as well as specific examples thereof, are intended to encompass both structural and functional equivalents thereof. Additionally, it is intended that such equivalents include both currently known equivalents and equivalents developed in the future, i.e., any elements developed that perform the same function, regardless of structure. The scope of the present invention, therefore, is not intended to be limited to the exemplary embodiments shown and described herein. Rather, the scope and spirit of present invention is embodied by the appended claims.

1. A method, comprising the steps of:
combining a reagent comprising a recombinant protein or C-terminal portion thereof with a sample;
maintaining the sample in vitro with the reagent therein under conditions to allow for amplification of a disease conformation of a native protein present in the sample thereby increasing the amount of any infectious protein in the sample.

2. The method of claim 1, further comprising:
assaying the sample for the presence of the disease conformation of the native protein.

3. The method of claim 1, wherein the sample comprises brain tissue.

4. The method of claim 3, wherein the brain tissue is from an animal chosen from a human, cow, pig, sheep, deer or chicken.

5. The method of claim 1, wherein the sample comprises material extracted from a first human patient for use in treating a second human patient.

6. The method of claim 1, wherein the sample comprises human blood or a component of the blood.

7. The method of claim 1, wherein the reagent comprises a full length recombinant protein having an amino acid sequence substantially identical to the amino acid sequence of the disease conformation of the native protein.

8. The method of claim 1, wherein the reagent comprises a recombinantly produced amino acid sequence comprising 50% or more of the C-terminal end of the amino acid sequence of the disease conformation of the native protein.

9. The method of claim 1, wherein the reagent comprises a plurality of different recombinantly produced amino acid sequences which sequences have substantial identity with a portion of the amino acid sequence of the disease conformation of the native protein.

10. The method of claim 4, wherein the conditions comprise maintaining a temperature within ±10° C. of the normal body temperature of the animal and wherein the reagent is cell free.

11. The method of claim 10, wherein the conditions comprise maintaining a temperature within ±2° C. of the normal body temperature of the animal and wherein the reagent is cell free.

12. The method of claim 4, wherein the animal is a cow and the reagent comprise amino acids 90-231 of the cow PrP protein.

13. The method of any one of claims 4, 10, 11 and 12, wherein the animal is a cow and the reagent comprises one or more amino acid sequences beginning with amino acid from 60 to 100 and ending with amino acid from 190 to 231 of the cow PrP protein.

14. The method of any one of claims 4, 10, 11 and 12, wherein the animal is a human and the reagent comprises one or more amino acid sequences beginning with amino acid from 60 to 100 and ending with amino acid from 190 to 231 of the human PrP protein.

15. The method of claim 14, wherein the sample comprises material extracted from a first human for use in treating a second human and the conditions comprise maintaining a temperature of 37° C.±2° C.

16. The method of claim 1, wherein the native protein is a PrP protein.

17. The method of claim 2, wherein the assaying is carried out within 20 hours or less after combining the reagent with the sample.

18. The method of claim 17, wherein the assaying is carried out within 5 hours or less after combining the reagent with the sample.

19. The method of claim 17, wherein the assaying is carried out within 2 hours or less after combining the reagent with the sample.

20. The method of claim 17, wherein the assaying is carried out within 1 hour or less after combining the reagent with the sample.

21. A method of assaying a sample comprising the steps of:
combining a reagent comprising a recombinant PrP protein or portion thereof with a sample suspected of containing an infectious PrP protein;
maintaining the sample in vitro with the reagent for twenty hours or less to allow for amplification of infectious PrP protein in the sample; and
assaying the sample for infectious PrP protein.

22. The method of claim 21, wherein the sample comprises brain tissue.

23. The method of claim 22, wherein the brain tissue is from an animal chosen from a human, cow, pig, sheep, deer or chicken.

24. The method of claim 21, wherein the assaying is carried out within 5 hours or less after combining the reagent with the sample.

25. The method of claim 24, wherein the assaying is carried out within 2 hours or less after combining the reagent with the sample.

26. The method of claim 25, wherein the assaying is carried out within 1 hour or less after combining the reagent with the sample.

27. The method of claim 25, wherein the portion thereof comprises 50% or more of the C-terminal end of a PrP protein.

28. The method of claim 27, wherein the portion thereof comprises 75% or more of the C-terminal end of a PrP protein.

29. The method of claim 27, wherein the portion thereof comprises 90% or more of the C-terminal end of a PrP protein.
